SEQUENCE LISTING (5 sequences)

SEQ ID NO: 1 - protein, 15 aa, Artificial Sequence
  Breast cancer specific peptide
KSSREGGGDQPDREQ

SEQ ID NO: 2 - protein, 15 aa, Artificial Sequence
  breast cancer specific peptide
QEPTNQKEDPIQLSL

SEQ ID NO: 3 - protein, 15 aa, Artificial Sequence
  breast cancer specific peptide
DQETATKMKVFIMQC

SEQ ID NO: 4 - protein, 15 aa, Artificial Sequence
  breast cancer specific peptide
SVAATTWSTWWAVCG

SEQ ID NO: 5 - protein, 15 aa, Artificial Sequence
  breast cancer specific peptide
NAEKRTDSGSRVMSD

(19) United States
(12) Patent Application Publication
(10) Pub. No.: US 2021/0199654 A1
(43) Pub. Date: Jul. 1, 2021

(54) ARRAY AND METHOD OF USE

(71) Applicant: Arizona Board of Regents on behalf of Arizona State University, Scottsdale, AZ (US)

(72) Inventor: Stephen Albert Johnston, Tempe, AZ (US)

(21) Appl. No.: 17/133,134

(22) Filed: Dec. 23, 2020

Related U.S. Application Data
(60) Provisional application No. 62/956,036, filed on Dec. 31, 2019.

(51) Int. Cl. G01N 33/563; C07K 7/08

(52) U.S. Cl. CPC G01N 33/563; G01N 2800/52; G01N 2800/54; C07K 7/08

(57) ABSTRACT

A method of producing a diagnostic peptide array, a diagnostic peptide array produced therefrom and methods of using a diagnostic peptide array are disclosed. Also disclosed, is a method of detecting an antibody associated with a disease of condition, including: contacting a biological sample with a diagnostic peptide array; and detecting the binding of one or more antibodies to a peptide that is associated with a disease or condition of interest, thereby detecting the antibody associated with a disease of condition.

### CROSS REFERENCE TO RELATED APPLICATION

This application claims the benefit of U.S. Provisional Application No. 62/956,036, filed Dec. 31, 2019, which is hereby expressly incorporated by reference in its entirety.

### SEQUENCE LISTING

The instant application contains a Sequence Listing which has been submitted electronically in ASCII format and is hereby incorporated by reference in its entirety. Said ASCII copy, created on Dec. 23, 2020, is named CALV.014A.txt and is 1.52 Kb in size.

### FIELD

This disclosure relates to diagnostic peptide arrays and in particular, to methods of producing a diagnostic genome peptide array and uses thereof.

### BACKGROUND

One of the great challenges in modem medicine is cost effectively detecting and monitoring diseases and conditions. Early detection of a condition can have a significant impact in the outcome of a disease, and yet, for most conditions, no single test exists that can detect disease before the appearance of major symptoms. Numerous groups have attempted to develop assays that can diagnose specific conditions; however such assays are limited to a specific disease or diagnosis. Moreover, monitoring health over a period of time is cost and time-prohibitive with currently available diagnostic assays.

### SUMMARY

Disclosed herein is a method of producing a diagnostic peptide array. In some embodiments, a diagnostic peptide array is produced by translating genomic sequences into one or more peptides and then synthesizing the one or more peptides in situ on silica or glass wafers. It is contemplated that the disclosed methods can utilize genome sequences to create Genome Peptide Arrays (GPA). In particular, the wafer surface is prepared to make GPA with optimal (close) space between the peptides. The genomic sequences can be from any organism and translated in any frame. GPA do not use sequences that encode natural proteins. Natural protein sequences are highly constrained in sequence use. The GPA sequences are not normally made, not subject to selection and therefore resemble random sequences. However, because of codon usage bias, the GPA sequences will represent an amino acid bias closer to natural sequences than random sequences. It is believed that this will match the antibody fitting better than random peptides. The GPA sequences are not conserved. The GPA arrays differ from Immunosignature arrays in that the peptides are not chosen from random sequence space, but the alternative frames in genomes. It was surprising to the inventors that another source of peptides besides random space could be used. The GPA arrays differ from Frameshift arrays in that the peptides are close together, such as less than or equal to 3 nm of each other to generate avidity, where Frameshift array peptides are spaced further apart to only allow high-affinity cognate peptide binding.

In some embodiments, the generated arrays are then developed as diagnostic platforms. For example, a dilution of sera or blood or other antibody containing fluid is applied to the arrays and the antibodies detected with a secondary antibody. The bound antibodies create a signature for that health state. Comparing healthy and subjects with a particular disease reveals a signature for that disease which can then be applied as a diagnostic. It is imperative to position peptides to be close enough in proximity to one another on the substrate, such as a wafer, to create an avidity effect to retain them on the substrate during washing. In some examples, the peptides are less than or equal to 3 nm of each other on the substrate to create an avidity effect to retain them on the substrate during washing. In some embodiments, a substrate, such as a wafer, is synthesized using hundreds of masks, such as up to 300 masks.

The peptides can be pre-synthesized by standard butyloxycarbonyl (BOC) or fluorenylmethyloxycarbonyl (FMOC) solid phase peptide synthesis approaches and then spotted on glass or other slides. For higher number of peptides (e.g., greater than 10,000) the peptides are synthesized in situ. This could be done with photoactivatable amino acids as done by Nimble Therapeutics (maskless photolithography) or PEPperPRINT. A standard mask-based system (much like Intel uses to lay down circuits) can be combined with BOC or FMOC peptide chemistry. Because 20 masks are required at each level of synthesis, 15 amino acid peptides require 300 masks. The inventors are the first to demonstrate that such a synthesis can be accomplished. In particular, prior to the present disclosure, it was not known that a 300 mask synthesis with less than 20 μm features could be accomplished.

It is contemplated that the disclosed arrays can be applicable to any disease, including cancer, such as Breast cancer, Valley Fever and Lyme disease.

A diagnostic peptide array produced from the one or more peptides for an antibody of interest, wherein the presence of the antibody of interest identifies a subject as having the disease or condition of interest.

A method of detecting an antibody associated with a disease or condition. The method including contacting a biological sample with a diagnostic peptide array and detecting the binding of one or more antibodies to a peptide in the diagnostic peptide that is associated with a disease or condition of interest, thereby detecting the antibody associated with a disease or condition.

Some embodiments provided herein relate to methods of producing a diagnostic peptide array. In some embodiments, the methods include translating one or more genomic non-protein encoding sequences into one or more peptides; and synthesizing one or more peptides onto a substrate. In some embodiments, the one or more peptides are arranged on the substrate to be equal to or less than 3 nm from each other. In some embodiments, the methods are used to produce a Genome Peptide Array (GPA). In some embodiments, the one or more peptides are a subset of those in the GPA. In some embodiments, the diagnostic peptide array comprises 400,000 or more peptides. In some embodiments, the one or more peptides is arranged on the substrate to equal about 0.5 nm to 3 nm from each other. In some embodiments, the method of producing comprises up to 300 masks. In some embodiments, the one or more peptides each contain 15 amino acids and all 20 amino acids are utilized in the one or more peptides. In some embodiments, the one or more peptides are arranged on the substrate to be about 60 peptides per nm².

Some embodiments provided herein relate to diagnostic arrays. In some embodiments, the diagnostic arrays are produced by the methods provided herein. For example, in some embodiments, the diagnostic arrays are produced by methods of producing a diagnostic peptide array. In some embodiments, the methods include translating one or more genomic non-protein encoding sequences into one or more peptides; and synthesizing one or more peptides onto a substrate. In some embodiments, the one or more peptides are arranged on the substrate to be equal to or less than 3 nm from each other. In some embodiments, the methods are used to produce a Genome Peptide Array (GPA). In some embodiments, the one or more peptides are a subset of those in the GPA. In some embodiments, the diagnostic peptide array comprises 400,000 or more peptides. In some embodiments, the one or more peptides is arranged on the substrate to equal about 0.5 nm to 3 nm from each other. In some embodiments, the method of producing comprises up to 300 masks. In some embodiments, the one or more peptides each contain 15 amino acids and all 20 amino acids are utilized in the one or more peptides. In some embodiments, the one or more peptides are arranged on the substrate to be about 60 peptides per nm².

Some embodiments provided herein relate to kits that include the diagnostic arrays as described herein. In some embodiments, the kits further include instructions for use.

Some embodiments provided herein relate to methods of detecting an antibody associated with a disease of condition. In some embodiments, the methods include contacting a biological sample obtained from a subject with a diagnostic peptide array of claim 9; and detecting the binding of one or more antibodies to a peptide that is associated with a disease or condition of interest, thereby detecting the antibody associated with a disease or condition. In some embodiments, the biological sample is serum sample. In some embodiments, the biological sample is a whole blood sample. In some embodiments, the methods are used to monitor efficacy of a treatment or reoccurrence. In some embodiments, the methods are used to diagnose a subject with cancer. In some embodiments, the cancer is selected from the group consisting Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor. In some embodiments, the breast cancer is stage 1 breast cancer. In some embodiments, the methods are used to diagnose Valley Fever. In some embodiments, the methods are used to diagnose Lyme disease.

The foregoing and other features of the disclosure will become more apparent from the following detailed description, which proceeds with reference to the accompanying figures.

### BRIEF DESCRIPTION OF THE DRAWINGS

FIG. 1 provides a comparison of the differences between the Genome Peptide (GPA), Frameshift (FSP), and Immunosignature (IMS) arrays.

FIG. 2 provides a Table summarizing the results of an IMS Array illustrating that the GPA has more chemical diversity than the standard array being used for immunosignatures.

FIGS. 3A-3C illustrate GPAs that can diagnose disease.

FIGS. 4A-4B demonstrate use of the GPAs to diagnose stage 1 breast cancer.

### DETAILED DESCRIPTION

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous, and are generally intended as “open” terms (e.g., the term “including” should be interpreted as “including but not limited to,” the term “having” should be interpreted as “having at least,” the term “includes” should be interpreted as “includes but is not limited to,” etc.).

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

To facilitate review of the various embodiments of this disclosure, the following explanations of specific terms are provided, along with particular examples:

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens, such as the peptides disclosed herein. The term “antigen” includes all related antigenic epitopes. “Epitope” or “antigenic determinant” refers to a site on an antigen to which B and/or T cells respond. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 amino acids in a unique spatial conformation. Methods of determining spatial conformation of epitopes include, for example, x-ray crystallography and 2-dimensional nuclear magnetic resonance.

Antibody: Immunoglobulin molecules and immunologically active portions of immunoglobulin molecules, i.e., molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complementarity determining region (CDR); and (vi) a F(ab′)2 fragment, a bivalent fragment comprising two Fab fragments linked by a disulfide bridge at the hinge region.

Immunoglobulins and certain variants thereof are known and many have been prepared in recombinant cell culture (e.g., see U.S. Pat. Nos. 4,745,055; 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984). Humanized antibodies and fully human antibodies are also known in the art.

Animal: Living multi-cellular vertebrate organisms, a category that includes, for example, mammals and birds. The term mammal includes both human and non-human mammals. Similarly, the term “subject” includes both human and veterinary subjects.

Array: An arrangement of molecules, such as biological macromolecules (such as peptides), in addressable locations on or in a substrate. In some examples, a substrate is a wafer, such as a wafer with multiple masks. A “microarray” is an array that is miniaturized so as to require or be aided by microscopic examination for evaluation or analysis. The array of molecules (“features”) makes it possible to carry out a very large number of analyses on a sample at one time. Within an array, each arrayed sample is addressable, in that its location can be reliably and consistently determined within at least two dimensions of the array. The feature application location on an array can assume different shapes. For example, the array can be regular (such as arranged in uniform rows and columns) or irregular. Thus, in ordered arrays the location of each sample is assigned to the sample at the time when it is applied to the array, and a key may be provided in order to correlate each location with the appropriate target or feature position. Often, ordered arrays are arranged in a symmetrical grid pattern, but samples can be arranged in other patterns (such as in radially distributed lines, spiral lines, or ordered clusters). Addressable arrays usually are computer readable, in that a computer can be programmed to correlate a particular address on the array with information about the sample at that position (such as hybridization or binding data, including for instance signal intensity). In some examples of computer readable formats, the individual features in the array are arranged regularly, for instance in a Cartesian grid pattern, which can be correlated to address information by a computer.

Binding or stable binding: An association between two substances or molecules, such as the association of an antibody with a peptide. Binding can be detected by any procedure known to one skilled in the art, such as by physical or functional properties of the formed complexes, such as a target/antibody complex.

Diagnostic: Identifying the presence or nature of a pathologic condition. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” means predicting the probability of development (for example, severity) of a pathologic condition.

Genomic non-protein encoding sequences: Stretches of DNA sequences that do not encode proteins (introns, non-coding DNA etc.) or when translated to peptides are not in the proteomic reading-frame (frameshifts).

Immune response: A response of a cell of the immune system, such as a B cell, T cell, or monocyte, to a stimulus. In one embodiment, the response is specific for a particular antigen (an “antigen-specific response”).

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

Label: A detectable compound or composition that is conjugated directly or indirectly to another molecule to facilitate detection of that molecule. Specific, non-limiting examples of labels include fluorescent tags, enzymatic linkages, and radioactive isotopes.

Peptide Modifications: Immunogenic peptides include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide of this disclosure is comprised of a sequence of amino acids, which may be either L- and/or D-amino acids, naturally occurring and otherwise.

Peptides can be modified by a variety of chemical techniques to produce derivatives having essentially the same activity as the unmodified peptides, and optionally having other desirable properties. For example, carboxylic acid groups of the protein, whether carboxyl-terminal or side chain, can be provided in the form of a salt of a pharmaceutically-acceptable cation or esterified to form a C₁-C₁₆ ester, or converted to an amide of formula NR₁R₂ wherein R₁ and R₂ are each independently H or C₁-C₁₆ alkyl, or combined to form a heterocyclic ring, such as a 5- or 6-membered ring. Amino groups of the peptide, whether amino-terminal or side chain, can be in the form of a pharmaceutically-acceptable acid addition salt, such as the HCl, HBr, acetic, benzoic, toluene sulfonic, maleic, tartaric and other organic salts, or can be modified to C₁-C₁₆ alkyl or dialkyl amino or further converted to an amide.

Hydroxyl groups of the peptide side chains may be converted to C₁-C₁₆ alkoxy or to a C₁-C₁₆ ester using well-recognized techniques. Phenyl and phenolic rings of the peptide side chains may be substituted with one or more halogen atoms, such as fluorine, chlorine, bromine or iodine, or with C₁-C₁₆ alkyl, C₁-C₁₆ alkoxy, carboxylic acids and esters thereof, or amides of such carboxylic acids. Methylene groups of the peptide side chains can be extended to homologous C₂-C₄ alkylenes. Thiols can be protected with any one of a number of well-recognized protecting groups, such as acetamide groups. Those skilled in the art will also recognize methods for introducing cyclic structures into the peptides of this disclosure to select and provide conformational constraints to the structure that result in enhanced stability.

Peptidomimetic and organomimetic embodiments are envisioned, whereby the three-dimensional arrangement of the chemical constituents of such peptido- and organomimetics mimic the three-dimensional arrangement of the peptide backbone and component amino acid side chains, resulting in such peptido- and organomimetics of an immunogenic polypeptide having measurable or enhanced ability to generate an immune response. For computer modeling applications, a pharmacophore is an idealized three-dimensional definition of the structural requirements for biological activity. Peptido- and organomimetics can be designed to fit each pharmacophore with current computer modeling software (using computer assisted drug design or CADD). See Walters, “Computer-Assisted Modeling of Drugs,” in Klegerman & Groves, eds., 1993, Pharmaceutical Biotechnology, Interpharm Press: Buffalo Grove, Ill., pp. 165-174 and Principles of Pharmacology, Munson (ed.) 1995, Ch. 102, for descriptions of techniques used in CADD. Also included are mimetics prepared using such techniques.

Peptide: Any chain of amino acids, regardless of length or post-translational modification (e.g., glycosylation or phosphorylation). A polypeptide can be between 3 and 30 amino acids in length. In one embodiment, a polypeptide is from about 5 to about 25 amino acids in length. In yet another embodiment, a polypeptide is from about 8 to about 12 amino acids in length. In yet another embodiment, a peptide is about 5 amino acids in length. With regard to polypeptides, the word “about” indicates integer amounts.

Sequence identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Within the context of an immunogenic peptide, a “conserved residue” is one which appears in a significantly higher frequency than would be expected by random distribution at a particular position in a peptide. In one embodiment, a conserved residue is one where the MHC structure may provide a contact point with the immunogenic peptide.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Homologs and variants of a polypeptide are typically characterized by possession of at least 75%, for example at least 80%, sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Suitable methods and materials for the practice or testing of this disclosure are described below. Such methods and materials are illustrative only and are not intended to be limiting. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999. In addition, the materials, methods, and examples are illustrative only and not intended to be limiting.

“Sequences in Life Space”: A phrase referring to a DNA/RNA sequence which is found in nature in an organism or virus. Some of these sequences encode natural proteins. Other sequences do not encode proteins.

### Description of Several Embodiments

### Introduction

Antibodies hold an immense amount of information about the health status of individuals. The challenge has been how to utilize this information in a simple, inexpensive way to allow monitoring health. Previously, immunosignatures (IMS) have been used as an approach to do this. In IMS, peptides were chosen from random sequence space and then synthesized close enough together to allow avidity to retain the antibodies. Immense random sequence space was chosen to allow limiting the number of synthesis steps, but had the disadvantage of not using all 20 amino acids and shorter length of peptides. IMS also did not reflect the amino acid bias in natural codons.

To solve this aforementioned problem, the inventor has developed the disclosed array and methods of use herein. The arrays and methods disclosed herein can utilize all 20 amino acids and use non-naturally coding life space rather than random space. In some embodiments, mask sets are designed to make GPAs by simply controlling the space between peptides.

As disclosed herein, a diagnostic peptide array is produced by translating genomic, but not natural protein coding, sequences into one or more peptides and then synthesizing the one or more peptides in situ on silica or glass wafers. For example, the method can include translating one or more genomic non-protein encoding sequences into one or more peptides and synthesizing one or more peptides onto a substrate. It is contemplated that the disclosed methods can utilize genome sequences to create Genome Peptide Arrays (GPA). In particular, the wafers are designed to make GPA by controlling the space between peptides. The GPAs are distinguished by the source of the peptides. In contrast to Frameshift peptides, GPA can include peptides that are not from exon mis-splicing in coding exons. The disclosed GPA are also distinct from immunosignatures for multiple reasons, including utilizing peptides with sequences in life space rather than from random sequence space (as seen with immunosignature peptides).

In some embodiments, the generated arrays are then developed as diagnostic platforms. For example, a dilution of sera or blood or other antibody containing fluid is applied to the arrays and the antibodies detected with a secondary antibody. The bound antibodies create a signature for that health state. Comparing healthy and subjects with a particular disease reveals a signature for that disease which can then be applied as a diagnostic. Since the peptides are mimotopes, not cognate epitopes, it is imperative to position peptides to be close enough in proximity to one another on the substrate, such as a wafer, to create an avidity effect to retain them on the substrate during washing. In some examples, the peptides are less than or equal to 3 nm of each other on the substrate to create an avidity effect to retain them on the substrate during washing. In some embodiments, a substrate, such as a wafer, is synthesized using up to 300 masks.

### Methods of Producing Peptide Arrays for the Detection of Disease or Condition

Disclosed herein is a method of producing a set of peptides for detecting one or more antibodies that are associated with one or more diseases or conditions of interest, for example as implemented in a peptide array. In some embodiments, method of producing a set of peptides for detecting one or more antibodies that are associated with one or more diseases or conditions of interest includes identifying a signature peptide profile for a disease or condition of interest, such as a set of informative peptides correlated to the disease or condition of interest, and translating the signature peptide profile to one or more high affinity peptides for an antibody of interest, wherein the presence of the antibody of interest identifies a subject as having a disease or condition of interest.

In embodiments, identifying a signature peptide profile includes: translating genomic sequences, after excluding those encoding native proteins, into one or more peptides and then synthesizing the one or more peptides in situ on silica or glass wafers. It is contemplated that the disclosed methods can utilize genome sequences from any organism to create Genome Peptide Arrays (GPA). In particular, mask sets are designed to make GPAs by controlling the space between peptides.

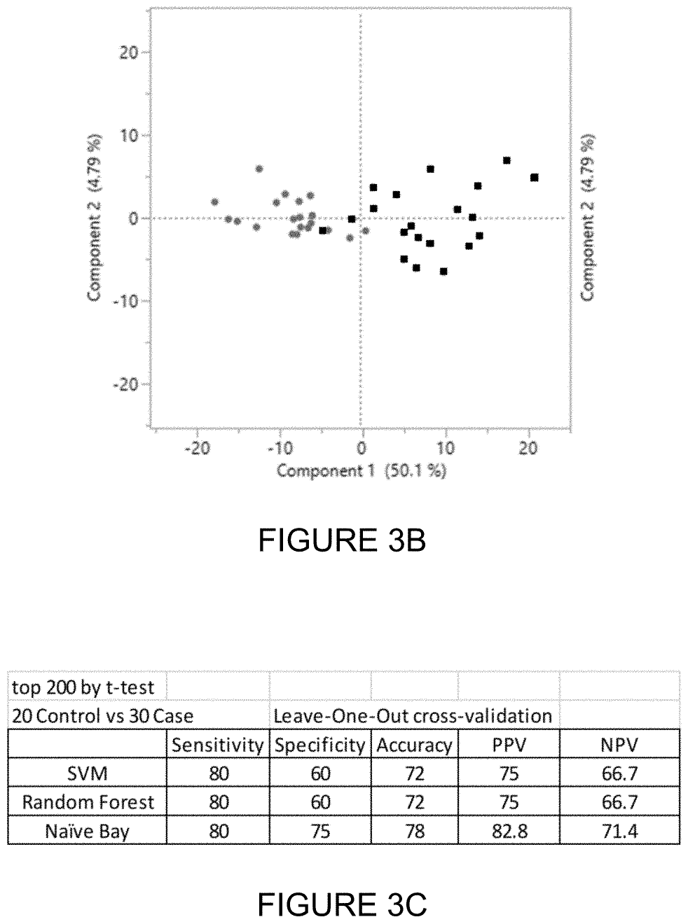

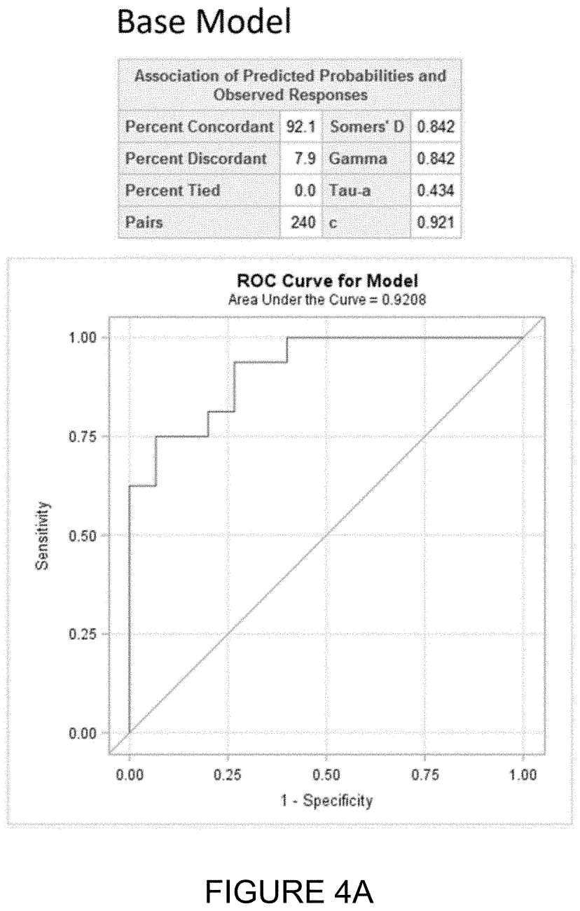

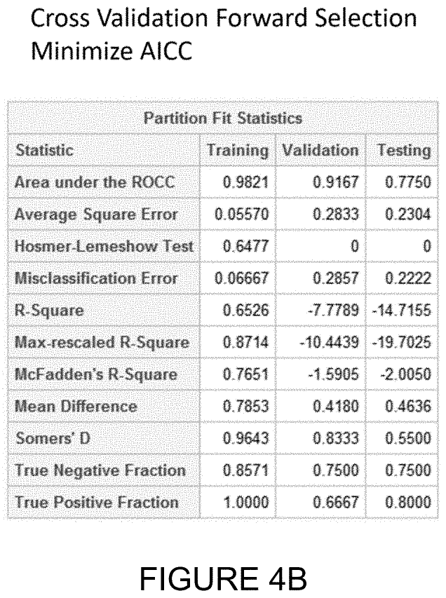

In comparing disease to non-disease samples, a large number of peptides that bind more antibody in disease versus non-disease samples or vise-versa can be identified. In embodiments, identifying differentially bound peptides includes identifying peptides on the peptide array that either bind less or more antibody in the profile as compared to the control. The control can be any suitable control. In one embodiment, the control comprises non-disease biological sample, such as sera, contacted with an identical array under the same experimental conditions. The control can be values taken from such a control, such that the control and test need not be conducted at the same time. Comparison of the disease immune profile to a normal control and identifying differentially bound peptides can be carried out via any suitable technique. FIGS. 3 and 4 as described below in the Example Section provides an example of this process applied to chronic Lyme disease and Breast Cancer.

In some examples, control sample is obtained from multiple individuals. For example sera obtained from healthy volunteers display a rather broad distribution of baseline binding reactivity, thus sample sera from a large number of non-diseased individuals may accommodate the population variability. In addition, signatures from sera of persons with a given disease are extremely consistent, unlike that of the non-disease sera. This observation implies that the immune system is constantly probing and reacting to local environments causing broad differences in signatures. However, once directed toward an antigen, antibodies tend to form a narrow and well-defined signature with little individual variability. In some embodiments, the control is a set of control values, for example an average or even weighted average, of the profile of several healthy individuals.

The binding of an antibody to a peptide array creates a pattern of binding that can be associated with a condition. The affinity of binding of an antibody to a peptide in the array can be mathematically associated with a condition. The binding pattern of an antibody to a plurality of different peptides of a peptide array can be mathematically associated with a condition. The avidity of binding of an antibody to a plurality of different peptides of a peptide array can be mathematically associated with a condition. This binding and avidity can comprise the interaction of an antibody in a biological sample with multiple, non-identical peptides in a peptide array. An avidity of binding of an antibody with multiple, non-identical peptides in a peptide array can determine an association constant of the antibody to the peptide array. In some embodiments, the concentration of an antibody in a sample contributes to an avidity of binding to a peptide array, for example, by trapping a critical number or antibodies in the array and allowing for rapid rebinding of an antibody to an array.

The avidity of binding of an antibody to a peptide array can be determined by a combination of multiple bond interaction. A cross-reactivity of an antibody to multiple peptides in a peptide array can contribute to an avidity of binding. In some embodiments, an antibody can recognize an epitope of about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 19 amino acids or about 20 amino acids. In some embodiments, a sequence of about 5 amino acids dominates a binding energy of an antibody to a peptide. The longer the peptide the more likely it will capture information from all antibodies. This is why extending the synthesis to 15 amino acids is a pivotal aspect of the technologies capabilities.

Off-target binding, and/or avidity, of an antibody to a peptide within a peptide array, for example, effectively compresses binding affinities that span femtomolar (fM) to micromolar (μM) dissociation constants into a range that can be quantitatively measured using only 3 logs of dynamic range. Avidity depends on the effective trapping of the antibody because the peptides are close enough together. An antibody can bind to a plurality of peptides in the array with association constants of 10³M⁻¹ or higher. An antibody can bind to a plurality of peptides in the array with association constants ranging from 10³ to 10⁶ M⁻¹, 2×10³ M⁻¹ to 10⁶ M⁻¹, and/or association constants ranging from 10⁴ M⁻¹ to 10⁶M⁻¹. An antibody can bind to a plurality of peptides in the array with a dissociation constant of about 1 fM, about 2 fM, about 3 fM, about 4 fM, about 5 fM, about 6 fM, about 7 fM, about 8 fM, about 9 fM, about 10 fM, about 20 fM, about 30 fM, about 40 fM, about 50 fM, about 60 fM, about 70 fM, about 80 fM, about 90 fM, about 100 fM, about 200 fM, about 300 fM, about 400 fM, about 500 fM, about 600 fM, about 700 fM, about 800 fM, about 900 fM, about 1 picomolar (pM), about 2 pM, about 3 pM, about 4 pM, about 5 pM, about 6 pM, about 7 pM, about 8 pM, about 9 pM, about 10 pM, about 20 pM, about 30 pM, about 40 pM, about 50 pM, about 60 pM, about 7 pM, about 80 pM, about 90 pM, about 100 pM, about 200 pM, about 300 pM, about 400 pM, about 500 pM, about 600 pM, about 700 pM, about 800 pM, about 900 pM, about 1 nanomolar (nM), about 2 nM, about 3 nM, about 4 nM, about 5 nM, about 6 nM, about 7 nM, about 8 nM, about 9 nM, about 10 nM, about 20 nM, about 30 nM, about 40 nM, about 50 nm, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, or about 100 μM.

An antibody can bind to a plurality of peptides in the array with a dissociation constant of at least 1 fM, at least 2 fM, at least 3 fM, at least 4 fM, at least 5 fM, at least 6 fM, at least 7 fM, at least 8 fM, at least 9 fM, at least 10 fM, at least 20 fM, at least 30 fM, at least 40 fM, at least 50 fM, at least 60 fM, at least 70 fM, at least 80 fM, at least 90 fM, at least 100 fM, at least 200 fM, at least 300 fM, at least 400 fM, at least 500 fM, at least 600 fM, at least 700 fM, at least 800 fM, at least 900 fM, at least 1 picomolar (pM), at least 2 pM, at least 3 pM, at least 4 pM, at least 5 pM, at least 6 pM, at least 7 pM, at least 8 pM, at least 9 pM, at least 10 pM, at least 20 pM, at least 30 pM, at least 40 pM, at least 50 pM, at least 60 pM, at least 7 pM, at least 80 pM, at least 90 pM, at least 100 pM, at least 200 pM, at least 300 pM, at least 400 pM, at least 500 pM, at least 600 pM, at least 700 pM, at least 800 pM, at least 900 pM, at least 1 nanomolar (nM), at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nm, at least 60 nM, at least 70 nM, at least 80 nM, at least 90 nM, at least 100 nM, at least 200 nM, at least 300 nM, at least 400 nM, at least 500 nM, at least 600 nM, at least 700 nM, at least 800 nM, at least 900 nM, at least 1 μM, at least 2 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, or about 100 μM.

A dynamic range of binding of an antibody from a biological sample to a peptide microarray can be described as the ratio between the largest and smallest value of a detected signal of binding. A signal of binding can be, for example, a fluorescent signal detected with a secondary antibody. Traditional assays are limited by pre-determined and narrow dynamic ranges of binding. The methods and arrays of the disclosure can detect a broad dynamic range of antibody binding to the peptides in the array. In some embodiments, a broad dynamic range of antibody binding can be detected on a logarithmic scale. In some embodiments, the methods and arrays of the disclosure allow the detection of a pattern of binding of a plurality of antibodies to an array using up to 2 logs of dynamic range, up to 3 logs of dynamic range, up to 4 logs of dynamic range or up to 5 logs of dynamic range.

As disclosed herein, the avidity effect declines when peptides are more than 3 nm apart. Using malemide labelling techniques, it is estimated that there are approximately 10 fM of peptide per 10 μm×10 μm feature of each individual peptide which yields approximately 60 peptides per nm².

Once the level of binding (usually by florescent intensity) of peptides bound by antibody are determined, the method further includes translating the signature peptide profile to high affinity peptides for an antibody of interest, wherein the presence of the antibody of interest identifies a subject as having a disease or condition of interest, for example to create a diagnostic peptide array for one or multiple diseases or conditions. High affinity antibodies can be used in more conventional assays, e.g., ELISAs. There are multiple approaches to constructing a set of high affinity peptides that bind an antibody of interest. In one embodiment, one or more antibodies of interest are affinity purified using the peptides they bind, for example, using the differentially bound peptides from the peptide array. In some embodiments, one or more antibodies of interest are identified by reverse engineering by using a peptide sequence, which may be graphed, for example by nucleic acid manipulation into an antibody scaffold. The antibody can then be used to probe the relevant cellular material to find the original antigen target, for example, by combining a pull down or Western blot with mass spectrometry. Once the target has been found, it can be used to determine the actual peptide sequence that induced the one or more antibodies of interest. In embodiments, a peptide, for example, a mimotope, is derived that has higher affinity for the informative antibody than an informative peptide from the array. This can be done by creating a library of variants on one or more antibodies of interest and selecting or screening for those with the highest affinity. This sequence does not have to correspond to the natural sequence of the inducing antigen, but is high affinity for the informative antibody. An advantage of this method is that the target antigen and/or epitope does not need to be identified. In embodiments, a peptide, for example, that has high affinity for the informative antibody is determined by informatic induction. For example, by aligning the sequences in the informative peptides potential epitope targets can be identified. These potential epitope targets can be matched against proteome sequences to find the original antigen and the natural high affinity peptide identified. Alternatively, the sequences of the potential epitope targets can be used as described above to define a high affinity peptide. However, in all of the methods described the goal is to assemble a collection of peptides that would bind all the antibodies that formed the original set of high affinity peptides for detecting one or more antibodies that are associated with one or more diseases or conditions of interest. These peptides would be high affinity to the informative antibody. Once the peptides for the antibody of interest are identified they can be used to create a diagnostic peptide array. Any number of peptides or disease and conditions may be used or detected with such a peptide array. In certain embodiments, each one of the peptides in the diagnostic array specifically binds an antibody with high affinity. In some instances, sensitivity of binding to the signature peptides can be enhanced by the buffering effect of the non-signature peptides. As such, in some cases, it may be determined not to reduce the array to only the signature peptides.

Any suitable peptide array can be used for an array on which the peptides are immobilized to a substrate, including discovery arrays and/or diagnostic arrays. In some embodiments, the array comprises between 500-1,000,000 peptides; between 500-500,000 peptides; between 500-250,000 peptides; between 500-100,000 peptides; between 500-50,000 peptides; or between 500-10,000 peptides. In some embodiments, the peptides are 8-35, 12-35, 15-25, 10-30, or 9-25 amino acids in length. In the case of peptide arrays used to identify the set of low affinity peptides for detecting one or more antibodies that are associated with one or more diseases or conditions of interest in some embodiments, the amino acid sequences of the peptides are based on sequences in life space, but not naturally in proteins, rather than from random sequence space. The use of life space sequence peptides enables the use of all 20 amino acids and shorter length. Normal, mutated, post-translationally modified, and mimetic epitopes corresponding to any disease or organism can be screened on the same microarray. In some embodiments however, the peptide arrays are used to identify the set of peptides for detecting one or more antibodies that are associated with one or more diseases or conditions of interest. In all embodiments, the pattern of amino acids present in the array is pre-defined, and is not a randomly organized peptide array.

As used herein, the term “substrate” refers to any type of solid support to which the peptides are immobilized. Examples of substrates include, but are not limited to, microarrays; beads; columns; optical fibers; wipes; nitrocellulose; nylon; glass; quartz; diazotized membranes (paper or nylon); silicones; polyformaldehyde; cellulose; cellulose acetate; paper; ceramics; metals; metalloids; semiconductive materials; coated beads; magnetic particles; plastics such as polyethylene, polypropylene, and polystyrene; gel-forming materials; silicates; agarose; polyacrylamides; methylmethracrylate polymers; sol gels; porous polymer hydrogels; nanostructured surfaces; nanotubes (such as carbon nanotubes); and nanoparticles (such as gold nanoparticles or quantum dots). When bound to a substrate, the peptides can be directly linked to the support, or attached to the surface via a linker. Thus, the solid substrate and/or the peptides can be derivatized using methods known in the art to facilitate binding of the peptides to the solid support, so long as the derivitization does not eliminate detection of binding between the peptides and an antibody. In the present disclosure, the peptides need to be close enough to each other, such as within 3 nm or less to facilitate avidity.

Other molecules, such as reference or control molecules, can be optionally immobilized on the substrate as well. Methods for immobilizing various types of molecules on a variety of substrates are well known to those of skill in the art. A wide variety of materials can be used for the solid surface. A variety of different materials can be used to prepare the support to obtain various properties. For example, proteins (e.g., bovine serum albumin) or mixtures of macromolecules (e.g., Denhardt's solution) can be used to minimize non-specific binding, simplify covalent conjugation, and/or enhance signal detection.

The peptide arrays can be contacted with a biological sample under any suitable conditions to promote binding of antibodies in the biological sample to peptides immobilized on the array. Thus, the disclosed methods are not limited by any specific type of binding conditions employed. Such conditions will vary depending on the array being used, the type of substrate, the density of the peptides arrayed on the substrate, desired stringency of the binding interaction, and nature of the competing materials in the binding solution. In some embodiments, the conditions comprise a step to remove unbound antibodies from the addressable array. Determining the need for such a step, and appropriate conditions for such a step, are well within the level of skill in the art.

Similarly, any suitable detection technique can be used in the disclosed methods detecting binding of antibodies in the biological sample to peptides on the array to generate a disease immune profile; In one embodiment, any type of detectable label can be used to label antibodies on the array, including but not limited to radioisotope labels, fluorescent labels, luminescent labels, and electrochemical labels (i.e.: ligand labels with different electrode mid-point potential, where detection comprises detecting electric potential of the label). Alternatively, bound antibodies can be detected, for example, using a detectably labeled secondary antibody. Methods that directly the bound antibodies, such as plasmon surface resonance, can also be used.

A peptide array can comprise a plurality of different peptides patterns on a surface. A peptide array can comprise, for example, a single, a duplicate, a triplicate, a quadruplicate, a quintuplicate, a sextuplicate, a septuplicate, an octuplicate, a nonuplicate, and/or a decuplicate replicate of the different pluralities of peptides and/or molecules. In some embodiments, pluralities of different peptides are spotted or synthesized in replica on the surface of a peptide array. A peptide array can, for example, comprise a plurality of peptides homogenously distributed on the array. A peptide array can, for example, comprise a plurality of peptides heterogeneously distributed on the array.

An inter-peptide distance in a peptide array is the distance between each peptide in a peptide microarray. An inter-peptide distance can contribute to an off-target binding and/or to an avidity of binding of an antibody to an array. This distance can be determined by the number of synthesis initiation sites per area. An intra-amino acid difference can be about 0.5 nm, about 1 nm, about 1 nm, 1.1 nm, about 1.2 nm, about 1.3 nm, about 1.4 nm, about 1.5 nm, about 1.6 nm, about 1.7 nm, about 1.8 nm, about 1.9 nm, about 2 nm, about 2.1 nm, about 2.2 nm, about 2.3 nm, about 2.4 nm, about 2.5 nm, about 2.6 nm, about 2.7 nm, about 2.8 nm, about 2.9 nm, about 3 nm. In some embodiments, the inter-peptide difference is between about 0.5 nm and about 3 nm.

An inter-peptide difference can be at least 0.5 nm, at least 1 nm, at least 1 nm, at least 1.1 nm, at least 1.2 nm, at least 1.3 nm, at least 1.4 nm, at least 1.5 nm, at least 1.6 nm, at least 1.7 nm, at least 1.8 nm, at least 1.9 nm, at least 2 nm, at least 2.1 nm, at least 2.2 nm, at least 2.3 nm, at least 2.4 nm, at least 2.5 nm, at least 2.6 nm, at least 2.7 nm, at least 2.8 nm, at least 2.9 nm, at least 3 nm.

An inter-peptide difference can be no more than 3 nm, not more than 3.1 nm, not more than 3.2 nm, not more than 3.3 nm, not more than 3.4 nm, not more than 3.5 nm, not more than 3.6 nm, not more than 3.7 nm, not more than 3.8 nm, not more than 3.9 nm, not more than 4 nm, not more than 4.1 nm, not more than 4.2 nm, not more than 4.3 nm, not more than 4.4 nm, not more than 4.5 nm, not more than 4.6 nm, not more than 4.7 nm, not more than 4.8 nm, not more than 4.9 nm, not more than 5 nm, not more than 5.1 nm, not more than 5.2 nm, not more than 5.3 nm, not more than 5.4 nm, not more than 5.5 nm, not more than 5.6 nm, not more than 5.7 nm, not more than 5.8 nm, not more than 5.9 nm, and/or not more than 6 nm. In some embodiments, the intra-amino acid distance is not more than 6 nanometers (nm).

An inter-peptide difference can range from 0.5 nm to 1 nm, 0.5 nm to 2 nm, 0.5 nm to 3 nm, or 0.5 nm to 3 nm.

A peptide can be “spotted” in a peptide array. A peptide spot can have various geometric shapes, for example, a peptide spot can be round, square, rectangular, and/or triangular. A peptide spot can have a plurality of diameters. Non-limiting examples of peptide spot diameters are about 3 μm to about 8 μm, about 3 to about 10 mm, about 5 to about 10 mm, about 10 μm to about 20 μm, about 30 μm, about 40 μm, about 50 μm, about 60 μm, about 70 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, and/or about 250 μm.

A peptide array can comprise a number of different peptides. In some embodiments, a peptide array comprises about 10 peptides, about 50 peptides, about 100 peptides, about 200 peptides, about 300 peptides, about 400 peptides, about 500 peptides, about 750 peptides, about 1000 peptides, about 1250 peptides, about 1500 peptides, about 1750 peptides, about 2,000 peptides; about 2,250 peptides; about 2,500 peptides; about 2,750 peptides; about 3,000 peptides; about 3,250 peptides; about 3,500 peptides; about 3,750 peptides; about 4,000 peptides; about 4,250 peptides; about 4,500 peptides; about 4,750 peptides; about 5,000 peptides; about 5,250 peptides; about 5,500 peptides; about 5,750 peptides; about 6,000 peptides; about 6,250 peptides; about 6,500 peptides; about 7,500 peptides; about 7,725 peptides 8,000 peptides; about 8,250 peptides; about 8,500 peptides; about 8,750 peptides; about 9,000 peptides; about 9,250 peptides; about 10,000 peptides; about 10,250 peptides; about 10,500 peptides; about 10,750 peptides; about 11,000 peptides; about 11,250 peptides; about 11,500 peptides; about 11,750 peptides; about 12,000 peptides; about 12,250 peptides; about 12,500 peptides; about 12,750 peptides; about 13,000 peptides; about 13,250 peptides; about 13,500 peptides; about 13,750 peptides; about 14,000 peptides; about 14,250 peptides; about 14,500 peptides; about 14,750 peptides; about 15,000 peptides; about 15,250 peptides; about 15,500 peptides; about 15,750 peptides; about 16,000 peptides; about 16,250 peptides; about 16,500 peptides; about 16,750 peptides; about 17,000 peptides; about 17,250 peptides; about 17,500 peptides; about 17,750 peptides; about 18,000 peptides; about 18,250 peptides; about 18,500 peptides; about 18,750 peptides; about 19,000 peptides; about 19,250 peptides; about 19,500 peptides; about 19,750 peptides; about 20,000 peptides; about 20,250 peptides; about 20,500 peptides; about 20,750 peptides; about 21,000 peptides; about 21,250 peptides; about 21,500 peptides; about 21,750 peptides; about 22,000 peptides; about 22,250 peptides; about 22,500 peptides; about 22,750 peptides; about 23,000 peptides; about 23,250 peptides; about 23,500 peptides; about 23,750 peptides; about 24,000 peptides; about 24,250 peptides; about 24,500 peptides; about 24,750 peptides; about 25,000 peptides; about 25,250 peptides; about 25,500 peptides; about 25,750 peptides; and/or about 30,000 peptides.

In some embodiments, a peptide array used in the methods and devices herein comprises more than 30,000 peptides. In some embodiments, a peptide array used in a method of health monitoring comprises about 330,000 peptides. In some embodiments the array comprise about 30,000 peptides; about 35,000 peptides; about 40,000 peptides; about 45,000 peptides; about 50,000 peptides; about 55,000 peptides; about 60,000 peptides; about 65,000 peptides; about 70,000 peptides; about 75,000 peptides; about 80,000 peptides; about 85,000 peptides; about 90,000 peptides; about 95,000 peptides; about 100,000 peptides; about 105,000 peptides; about 110,000 peptides; about 115,000 peptides; about 120,000 peptides; about 125,000 peptides; about 130,000 peptides; about 135,000 peptides; about 140,000 peptides; about 145,000 peptides; about 150,000 peptides; about 155,000 peptides; about 160,000 peptides; about 165,000 peptides; about 170,000 peptides; about 175,000 peptides; about 180,000 peptides; about 185,000 peptides; about 190,000 peptides; about 195,000 peptides; about 200,000 peptides; about 210,000 peptides; about 215,000 peptides; about 220,000 peptides; about 225,000 peptides; about 230,000 peptides; about 240,000 peptides; about 245,000 peptides; about 250,000 peptides; about 255,000 peptides; about 260,000 peptides; about 265,000 peptides; about 270,000 peptides; about 275,000 peptides; about 280,000 peptides; about 285,000 peptides; about 290,000 peptides; about 295,000 peptides; about 300,000 peptides; about 305,000 peptides; about 310,000 peptides; about 315,000 peptides; about 320,000 peptides; about 325,000 peptides; about 330,000 peptides; about 335,000 peptides; about 340,000 peptides; about 345,000 peptides; about 350,000 peptides; about 360,000 peptides; about 370,000 peptides; about 380,000 peptides; about 390,000 peptides; about 400,000 peptides; about 405,000 peptides; about 408,000 peptides and/or about 410,000 peptides. In some embodiments, a peptide array used in a method of health monitoring comprises more than 330,000 peptides, more than 350,000 peptides, more than 400,000 peptides such as between 350,000 and 410,000 peptides, or 400,000 and 410,000 peptides.

A peptide array can comprise a number of different peptides. In some embodiments, a peptide array comprises at least 2,000 peptides; at least 3,000 peptides; at least 4,000 peptides; at least 5,000 peptides; at least 6,000 peptides; at least 7,000 peptides; at least 8,000 peptides; at least 9,000 peptides; at least 10,000 peptides; at least 11,000 peptides; at least 12,000 peptides; at least 13,000 peptides; at least 14,000 peptides; at least 15,000 peptides; at least 16,000 peptides; at least 17,000 peptides; at least 18,000 peptides; at least 19,000 peptides; at least 20,000 peptides; at least 21,000 peptides; at least 22,000 peptides; at least 23,000 peptides; at least 24,000 peptides; at least 25,000 peptides; at least 30,000 peptides; at least 40,000 peptides; at least 50,000 peptides; at least 60,000 peptides; at least 70,000 peptides; at least 80,000 peptides; at least 90,000 peptides; at least 100,000 peptides; at least 110,000 peptides; at least 120,000 peptides; at least 130,000 peptides; at least 140,000 peptides; at least 150,000 peptides; at least 160,000 peptides; at least about 170,000 at least 180,000 peptides; at least 190,000 peptides; at least 200,000 peptides; at least 210,000 peptides; at least 220,000 peptides; at least 230,000 peptides; at least 240,000 peptides; at least 250,000 peptides; at least 260,000 peptides; at least 270,000 peptides; at least 280,000 peptides; at least 290,000 peptides; at least 300,000 peptides; at least 310,000 peptides; at least 320,000 peptides; at least 330,000 peptides; at least 340,000 peptides; at least 350,000 peptides. In some embodiments, a peptide array used in a method of health monitoring comprises at least 330,000 peptides. In some embodiments more than 400,000 peptides are used on the array.

A peptide can be physically tethered to a peptide array by a linker molecule. The N- or the C-terminus of the peptide can be attached to a linker molecule. A linker molecule can be, for example, a functional plurality or molecule present on the surface of an array, such as an imide functional group, an amine functional group, a hydroxyl functional group, a carboxyl functional group, an aldehyde functional group, and/or a sulfhydryl functional group. A linker molecule can be, for example, a polymer. In some embodiments the linker is maleimide. In some embodiments the linker is a glycine-serine-cysteine (GSC) or glycine-glycine-cysteine (GGC) linker. In some embodiments, the linker consists of a polypeptide of various lengths or compositions. In some cases the linker is polyethylene glycol of different lengths. In yet other cases, the linker is hydroxymethyl benzoic acid, 4-hydroxy-2-methoxy benzaldehyde, 4-sulfamoyl benzoic acid, or other suitable for attaching a peptide to the solid substrate.

A surface of a peptide array can comprise a plurality of different materials. A surface of a peptide array can be, for example, glass. Non-limiting examples of materials that can comprise a surface of a peptide array include glass, functionalized glass, silicon, germanium, gallium arsenide, gallium phosphide, silicon dioxide, sodium oxide, silicon nitrade, nitrocellulose, nylon, polytetraflouroethylene, polyvinylidendiflouride, polystyrene, polycarbonate, methacrylates, or combinations thereof.

A surface of a peptide array can be flat, concave, or convex. A surface of a peptide array can be homogeneous and a surface of an array can be heterogeneous. In some embodiments, the surface of a peptide array is flat.

A surface of a peptide array can be coated with a coating. A coating can, for example, improve the adhesion capacity of an array. A coating can, for example, reduce background adhesion of a biological sample to an array. In some embodiments, a peptide array comprises a glass slide with an aminosilane-coating.

A peptide array can have a plurality of dimensions. A peptide array can be a peptide microarray.

Binding interactions between components of a sample and an array can be detected in a variety of formats. In some formats, components of the samples are labeled. The label can be a radioisotype or dye among others. The label can be supplied either by administering the label to a patient before obtaining a sample or by linking the label to the sample or selective component(s) thereof

Binding interactions can also be detected using a secondary detection reagent, such as an antibody. For example, binding of antibodies in a sample to an array can be detected using a secondary antibody specific for the isotype of an antibody (e.g., IgG (including any of the subtypes, such as IgG1, IgG2, IgG3 and IgG4), IgA, IgM). The secondary antibody is usually labeled and can bind to all antibodies in the sample being analyzed of a particular isotype. Different secondary antibodies can be used having different isotype specificities. Although there is often substantial overlap in compounds bound by antibodies of different isotypes in the same sample, there are also differences in profile.

Binding interactions can also be detected using label-free methods, such as surface plasmon resonance (SPR) and mass spectrometry. SPR can provide a measure of dissociation constants, and dissociation rates. The A-100 Biocore/GE instrument, for example, is suitable for this type of analysis. FLEXchips can be used to analyze up to 400 binding reactions on the same support.

Disclosed are peptide arrays produced by the methods described herein as well as kits including such peptide arrays.

### Methods of Detecting a Disease or Condition

The present disclosure provides peptides arrays for the use of medical diagnostics, for example to detect a disease and/or condition in sample, such as a sample obtained from a subject. In some embodiments, the peptide array may be used in determining response to administration of drugs or vaccines. In embodiments, a method of detecting an antibody associated with a disease of condition, includes contacting a biological sample with a diagnostic peptide array, and detecting the binding of one or more antibodies to a peptide that is associated with a disease or condition of interest, thereby detecting the antibody associated with a disease of condition. A condition that can be diagnosed or prognosed with a peptide array includes, for example, cancer, autoimmune disorder, an infectious disease, an epidemic, transplant rejection, a metabolic disease, a cardiovascular disease, a dermatological disease, a hematological disease, a neurodegenerative disease, an inflammatory disease, and infarctions (e.g. myocardial infarction, stroke).

The binding of a molecule to an array of the invention creates a pattern of binding that can be associated with a condition. The affinity of binding of a molecule to a peptide in the array can be mathematically associated with a condition. The off-target binding pattern of an antibody to a plurality of different peptides of the invention can be mathematically associated with a condition. The avidity of binding of a molecule to a plurality of different peptides of the invention can be mathematically associated with a condition. The off-target binding and avidity can comprise the interaction of a molecule in a biological sample with multiple, non-identical peptides in a peptide array. An avidity of binding of a molecule with multiple, non-identical peptides in a peptide array can determine an association constant of the molecule to the peptide array. In some embodiments, the concentration of an antibody in a sample contributes to an avidity of binding to a peptide array, for example, by trapping a critical number or antibodies in the array and allowing for rapid rebinding of an antibody to an array.

A diagnostic peptide array of the present disclosure can be used to diagnose or prognose cancers including, for example, prostate cancer, lung cancer, colon cancer, bladder cancer, brain cancer, breast cancer, esophageal cancer, Hodgkin lymphoma, kidney cancer, larynx cancer, leukemia, liver cancer, melanoma of the skin, myeloma, non-Hodgkin lymphoma, oral cavity cancer, ovarian cancer, pancreatic cancer, rectal cancer, stomach cancer, testicular cancer, thyroid cancer, urinary bladder cancer, and cervical cancer.

A diagnostic peptide array of the present disclosure can be used to diagnose or prognose cancers including epidemics caused by, for example, viruses, bacteria, or parasites, or non-infectious agents.

A diagnostic peptide array of the present disclosure can be used to diagnose or prognose metabolic disease including, for example, abetalipoproteinemia, adrenoleukodystrophy (ALD), crigler-najjar syndrome, cystinuria, hartnup disease, histidinemia, Menkes disease, phenylketonuria (PKU), sitosterolemia, Smith-Lemli-Opiz syndrome, tyrosinemia type I, urea cycle disorders, Wilson's disease, Zellweger syndrome, maple syrup urine disease (MSUD; branched-chain ketoaciduria), glycogen storage disease, glutaric acidemia type 1, alcaptonuria, medium chain acyl dehydrogenase deficiency (glutaric acidemia type 2), acute intermittent porphyria, Lesch-Nhyhan syndrome, congenital adrenal hyperplasia, Kearns-Sayre syndrome, Gaucher's disease, diabetes (type 1), hereditary hemochromatosis, and Niemann-Pick disease.

A diagnostic peptide array of the present disclosure can be used to diagnose or prognose cardiovascular disease including, for example, angina, arrhythmia, atherosclerosis, cardiomyopathy, cerebrovascular accident (stroke), cerebrovascular disease, congenital heart disease, Jye Berghofer Syndrome, congestive heart failure, myocarditis, valve disease, coronary artery disease, dilated cardiomyopathy, diastolic dysfunction, endocarditis, high blood pressure (hypertension), hypertrophic cardiomyopathy, mitral valve prolapse, myocardial infarction, venous thromboembolism.

A diagnostic peptide array of the present disclosure can be used to diagnose or prognose dermatological disorders including, for example, acne, actinic keratosis, angioma, Athlete's foot, aquagenic pruritus, argyria, atopic dermatitis, baldness, basal cell carcinoma, bed sore, Behcet's disease, blepharitis, boil, Bowen's disease, bullous pemphigoid, canker sore, carbuncles, cellulitis, chloracne, chronic dermatitis of the hands and feet, cold sores, contact dermatitis (includes poison ivy, oak, sumac), creeping eruption, dandruff, dermatitis, dermatitis herpetiformis, dermatofibroma, diaper rash, dyshidrosis, eczema, epidermolysis bullosa, erysipelas, erythroderma, friction blister, genital wart, gestational pemphigoid, Grover's disease, hemangioma, Hidradenitis suppurativa, hives, hyperhidrosis, ichthyosis, impetigo, jock itch, Kaposi's sarcoma, keloid, keratoacanthoma, keratosis pilaris, Lewandowsky-Lutz dysplasia, lice infection, Lichen planus, Lichen simplex chronicus, lipoma, lymphadenitis, malignant melanoma, melasma, miliaria, molluscum contagiosum, nummular dermatitis, Paget's disease of the nipple, pediculosis, pemphigus, perioral dermatitis, photoallergy, photosensitivity, Pityriasis rosea, Pityriasis rubra pilaris, porphyria, psoriasis, Raynaud's disease, ringworm, rosacea, scabies, scleroderma, scrofula, sebaceous cyst, seborrheic keratosis, seborrhoeic dermatitis, shingles, skin cancer, skin tags, spider veins, squamous cell carcinoma, stasis dermatitis, sunburn, tick bite, tinea barbae, tinea capitis, tinea corporis, tinea cruris, tinea pedis, tinea unguium, tinea versicolor, tinea, tungiasis, urticaria (Hives), Vagabond's disease, vitiligo, warts, wheal (“weal” and “welt”).

A diagnostic peptide array of the present disclosure can be used to diagnose or prognose hematological disorders including, for example Anaphylactoid Purpura (Henock-Schonlein Disease), allergic purpura (Henock-Schonlein Disease), low red blood cells (anemia), hemolytic anemia, hypoproliferative anemia, macrocytic anemia, microcytic anemia, normocytic anemia, pernicious anemia (Vitamin B12 deficiency), basophilia, blood vessel abnormalities, dysfibrinogenemia, eosinophilia, erythrocytosis/polycythemia, essential thrombocythemia, excess platelets (thrombocytosis), excess red blood cells (erythrocytosis/polycythemia), excess white blood cells (leukocytosis), Factor V Leiden Mutation, fibrin clot formation abnormalities, folic acid deficiency, hemophilia, hereditary von Willebrand's Disease, inherited hypercoagulation disorders, inherited platelet abnormalities, iron deficiency, low platelets (thrombocytopenia), low white blood cells (neutropenia), lymphocytosis, myelofibrosis with myeloid metaplasia, monocytosis, myeloproliferative disorders, neutrophilia, platelet abnormalities, polycythemia vera, premalignant blood disorders, scurvy, Systemic Lupus Erythematosus (SLE), thrombocytopenia, and sickle cell disease.

A diagnostic peptide array of the present disclosure can be used to diagnose or prognose neurodegenerative diseases including, for example, alcoholism, Alexander's disease, Alper's disease, Alzheimer's disease, Amyotrophic lateral sclerosis, ataxia telangiectasia, Batten disease (also known as Spielmeyer-Vogt-Sjogren-Batten disease), bovine spongiform encephalopathy (BSE), Canavan disease, Cockayne syndrome, Corticobasal degeneration, Creutzfeldt-Jakob disease, Huntington's disease, HIV-associated dementia, Kennedy's disease, Krabbe's disease, Lewy body dementia, Machado-Joseph disease (Spinocerebellar ataxia type 3), multiple sclerosis, Multiple System Atrophy, narcolepsy, neuroborreliosis, Parkinson's disease, Pelizaeus-Merzbacher Disease, Pick's disease, primary lateral sclerosis, prion diseases, Refsum's disease, Sandhoffs disease, Schilder's disease, subacute combined degeneration of spinal cord secondary to pernicious anaemia, schizophrenia, spinocerebellar ataxia (multiple types with varying characteristics), spinal muscular atrophy, Steele-Richardson-Olszewski disease, and Tabes dorsalis.

A diagnostic peptide array of the present disclosure can be used to diagnose or prognose inflammatory diseases including, for example, asthma, autoimmune diseases, chronic inflammation, chronic prostatitis, glomerulonephritis, hypersensitivities, inflammatory bowel diseases, pelvic inflammatory disease, reperfusion injury, rheumatoid arthritis, transplant rejection, and vasculitis.

A diagnostic peptide array of the present disclosure can be used to diagnose or prognose a bacterial, viral or other infections, such as Lyme disease and Valley Fever.

In some embodiments, a method of the disclosure can be used as a method of diagnosing, monitoring, and treating a condition. A method of treating a condition can require the prescription of a therapeutic agent targeted to treat the subject's condition or disease. In some embodiments, a therapeutic agent can be prescribed in a range of from about 1 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

### Biological Samples

The methods and arrays disclosed herein allow for, for example, methods of detecting a disease and/or condition with small quantities of biological samples from a subject. In some embodiments, the biological samples can be used in a disclosed method without further processing and in small quantities. In some embodiments, the biological samples comprise, blood, serum, saliva, sweat, cells, tissues, or any bodily fluid. In some embodiments, about 0.5 nl, about 1 nl, about 2 nl, about 3 nl, about 4 nl, about 5 nl, about 6 nl, about 7 nl, about 8 nl, about 9 nl, about 10 nl, about 11 nl, about 12 nl, about 13 nl, about 14 nl, about 15 nl, about 16 nl, about 17 nl, about 18 nl, about 19 nl, about 20 nl, about 21 nl, about 22 nl, about 23 nl, about 24 nl, about 25 nl, about 26 nl, about 27 nl, about 28 nl, about 29 nl, about 30 nl, about 31 nl, about 32 nl, about 33 nl, about 34 nl, about 35 nl, about 36 nl, about 37 nl, about 38 nl, about 39 nl, about 40 nl, about 41 nl, about 42 nl, about 43 nl, about 44 nl, about 45 nl, about 46 nl, about 47 nl, about 48 nl, about 49 nl, or about 50 nl, about 51 nl, about 52 nl, about 53 nl, about 54 nl, about 55 nl, about 56 nl, about 57 nl, about 58 nl, about 59 nl, about 60 nl, about 61 nl, about 62 nl, about 63 nl, about 64 nl, about 65 nl, about 66 nl, about 67 nl, about 68 nl, about 69 nl, about 70 nl, about 71 nl, about 72 nl, about 73 nl, about 74 nl, about 75 nl, about 76 nl, about 77 nl, about 78 nl, about 79 nl, about 80 nl, about 81 nl, about 82 nl, about 83 nl, about 84 nl, about 85 nl, about 86 nl, about 87 nl, about 88 nl, about 89 nl, about 90 nl, about 91 nl, about 92 nl, about 93 nl, about 94 nl, about 95 nl, about 96 nl, about 97 nl, about 98 nl, about 99 nl, about 0.1, about 0.2 μl, about 0.3 μl, about 0.4 μl, about 0.5 μl, about 0.6 μl. about 0.7 about 0.8 μl, about 0.9 μl, about 1 μl, about 2 μl, about 3 about 4 μl, about 5 μl, about 6 about 7 μl, about 8 μl, about 9 μl, about 10 μl, about 11 μl, about 12 μl, about 13 μl, about 14 μl, about 15 μl, about 16 μl, about 17 μl, about 18 μl, about 19 μl, about 20 μl, about 21 μl, about 22 μl, about 23 μl, about 24 μl, about 25 μl, about 26 μl, about 27 μl, about about 29 μl, about 30 μl, about 31 μl, about 32 μl, about 33 μl, about 34 μl, about 35 μl, about 36 μl, about 37 μl, about 38 μl, about 39 μl, about 40 μl, about 41 μl, about 42 μl, about 43 μl, about 44 μl, about 45 μl, about 46 μl, about 47 μl, about 48 μl, about 49 μl, or about 50 μl of biological samples are required for analysis by an array.

A biological sample from a subject can be for example, collected from a subject and directly contacted with an array of the invention. In some embodiments, the biological sample does not require a preparation or processing step prior to being contacted with an array of the invention. In some embodiments, a dry blood sample from a subject is reconstituted in a dilution step prior to being contacted with an array of the invention. A dilution can provide an optimum concentration of an antibody from a biological sample of a subject for testing according to the methods disclosed herein.

In some embodiments, the disclosed methods require no more than about 0.5 nl to about 50 nl, no more than about 1 nl to about 100 nl, no more than about 1 nl to about 150 nl, no more than about 1 nl to about 200 nl, no more than about 1 nl to about 250 nl, no more than about 1 nl to about 300 nl, no more than about 1 nl to about 350 nl, no more than about 1 nl to about 400 nl, no more than about 1 to about 450 nl, no more than about 5 nl to about 500 nl, no more than about 5 nl to about 550 nl, no more than about 5 nl to about 600 nl, no more than about 5 nl to about 650 nl, no more than about 5 nl to about 700 nl, no more than about 5 nl to about 750 nl, no more than about 5 nl to about 800 nl, no more than about 5 nl to about 850 nl, no more than about 5 nl to about 900 nl, no more than about 5 nl to about 950 nl, no more than about 5 nl to about 1 μl, no more than about 0.5 μl to about 1 μl, no more than about 0.5 μl to about 5 μl, no more than about 1 μl to about 10 μl, no more than about 1 μl to about 20 μl, no more than about 1 μl to about 30 μl, no more than about 1 μμl to about 40 μl, or no more than about 1 μl to about 50 μl.

In some embodiments, the methods of the invention require at least 0.5 nl to about 50 nl, at least about 1 nl to about 100 nl, at least about 1 nl to about 150 nl, at least about 1 nl to about 200 nl, at least about 1 nl to about 250 nl, at least about 1 nl to about 300 nl, at least about 1 nl to about 350 nl, at least about 1 nl to about 400 nl, at least about 1 to about 450 nl, at least about 5 nl to about 500 nl, at least about 5 nl to about 550 nl, at least about 5 nl to about 600 nl, at least about 5 nl to about 650 nl, at least about 5 nl to about 700 nl, at least about 5 nl to about 750 nl, at least about 5 nl to about 800 nl, at least about 5 nl to about 850 nl, at least about 5 nl to about 900 nl, at least about 5 nl to about 950 nl, at least about 5 nl to about 1 μl, at least about 0.5 μl to about 1 μl, at least about 0.5 μl to about 5 μl, at least about 1 μl to about 10 μl, at least about 1 IA to about 20 μl, at least about 1 μl to about 30 μl, at least about 1 μl to about 40 μl, at least about 1 μl to about 50 μl, or at least 50 μl

In some embodiments, biological samples from a subject are too concentrated and require a dilution prior to being contacted with an array of the invention. A plurality of dilutions can be applied to a biological sample prior to contacting the sample with an array of the invention. A dilution can be a serial dilution, which can result in a geometric progression of the concentration in a logarithmic fashion. For example, a ten-fold serial dilution can be 1 M, 0.01 M, 0.001 M, and a geometric progression thereof. A dilution can be, for example, a one-fold dilution, a two-fold dilution, a three-fold dilution, a four-fold dilution, a five-fold dilution, a six-fold dilution, a seven-fold dilution, an eight-fold dilution, a nine-fold dilution, a ten-fold dilution, a sixteen-fold dilution, a twenty-five-fold dilution, a thirty-two-fold dilution, a sixty-four-fold dilution, and/or a one-hundred-and-twenty-five-fold dilution.

A biological sample can be derived from a plurality of sources within a subject's body and a biological sample can be collected from a subject in a plurality of different circumstances. A biological sample can be collected, for example, during a routine medical consultation, such as a blood draw during an annual physical examination. A biological sample can be collected during the course of a non-routine consultation, for example, a biological sample can be collected during the course of a biopsy. A subject can also collect a biological sample from oneself, and a subject can provide a biological sample to be analyzed by the methods and systems of the invention in a direct-to-consumer fashion. In some embodiments, a biological sample can be mailed to a provider of the methods and arrays of the invention. In some embodiments, a dry biological sample, such as a dry blood sample from a subject on a filter paper, is mailed to a provider of the methods and arrays of the invention.

### Kits

Provided by this disclosure are kits that can be used to diagnose a subject with a particular condition or disease and/or monitor the efficacy of a treatment or reoccurrence. Exemplary kits include at least one array, such as GPA. The disclosed kits can include instructional materials disclosing means of use of the array in the kit. The instructional materials can be written, in an electronic form (such as a computer diskette or compact disk) or can be visual (such as video files).

The following examples are provided to illustrate particular features of certain embodiments. However, the particular features described below should not be construed as limitations on the scope of the disclosure, but rather as examples from which equivalents will be recognized by those of ordinary skill in the art.

### EXAMPLE

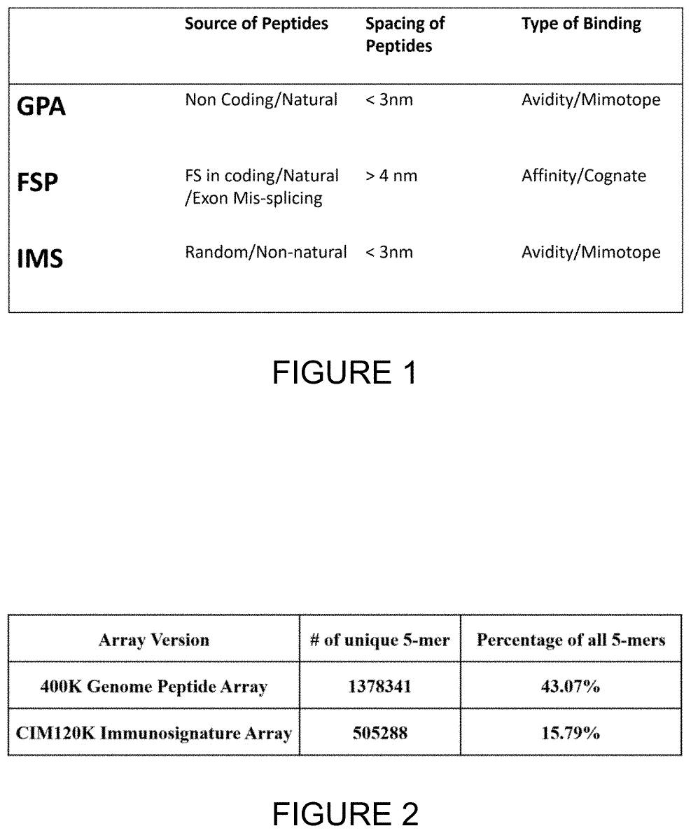

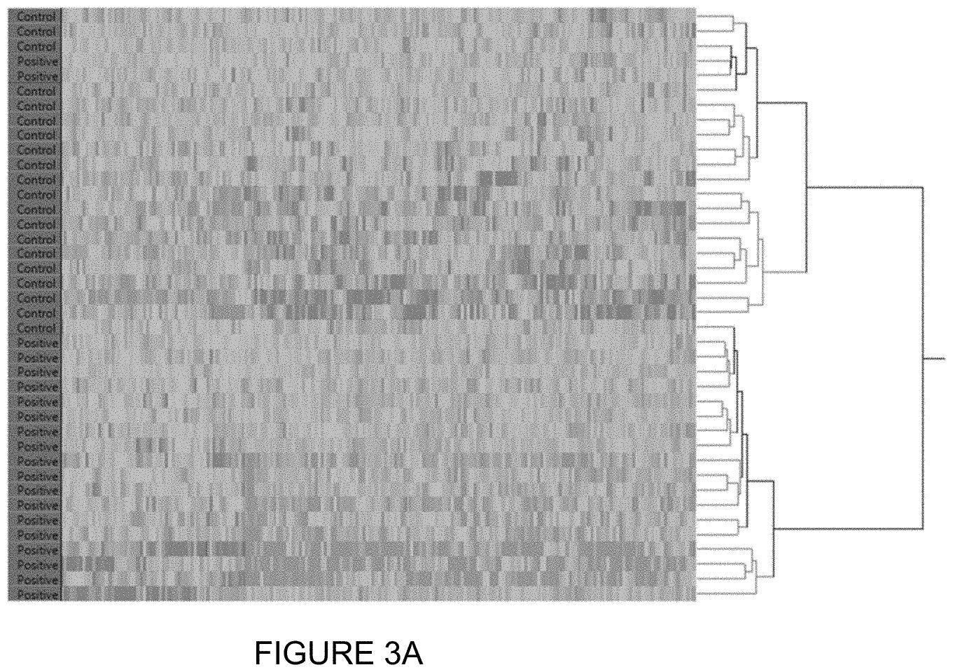

This example illustrates the differences between a GPA and other array technologies as well as uses of an exemplary GPA. First, FIG. 1 provides a table comparing the differences between the GPA, Frameshift and Immunosignature arrays. FIG. 2 provides a Table summarizing the results of GPA and Immunosignature (IMS) Array illustrating the GPA has more chemical diversity than the standard array being used for immunosignatures. The standard IMS array has 125K peptides averaging 12 amino acids (aa) in length. Only 16 of 20 amino acids (aa) are used to reduce the masks required. The peptides were chosen from random sequence space to maximize chemical diversity while at the same time minimizing the number of masks steps required. The IMS arrays use 64 masks. The GPA array was constructed to generate 15 amino acid peptides using all 20 amino acids. The GPA are predicted from the human genome as alternate reading frames. There are approximately 400K such peptides. They can be generated from any genome in nature, including plant and microbial. It requires 300 masks to synthesize 15 aa mers with 20 aa. The chemical complexity was measured as the percent of possible 5 aa sequences represented. There are 3.2M possible 5 aa mers using 20aa. As can be seen, the 400K GPA has approximately 3 times the diversity. Since it has approximately 3 times the number of peptides, the implication is that the arrays formed from alternate life space is as chemically diverse as from random space.

FIGS. 3A-3C demonstrate Genome Peptide Arrays that can diagnose disease. Twenty sera samples from human subjects with chronic Lyme disease and 20 samples from human subjects that did not have chronic Lyme disease (healthy subjects) were applied to the 400K GPA. The samples were processed by a standard procedure. After incubation the array was washed and a fluorescent secondary antibody was applied to detect the amount of primary antibody on each peptide feature. The arrays were laser scanned. The data was used to pick peptides that distinguished the infected from healthy samples. As evident, the GPAs were able to distinguish the infected samples with at least 70-80% accuracy. This is better than the current diagnostic for Lyme.

FIGS. 4A-4B demonstrate use of an exemplary GPA to diagnose stage 1 breast cancer. Sixteen blood samples were obtained from women diagnosed with stage 1 breast cancer and compared to 16 age matched samples from subjects that did not have breast cancer. The following five peptides distinguished between the two sets were chosen to estimate accuracy: KSSREGGGDQPDREQ (SEQ ID NO: 1); QEPTNQKEDPIQLSL (SEQ ID NO: 2); DQETATKMKVFIMQC (SEQ ID NO: 3); SVAATTWSTWWAVCG (SEQ ID NO: 4); and NAEKRTDSGSRVMSD (SEQ ID NO: 5). A blinded test set was not used.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims. We therefore claim as our invention all that comes within the scope and spirit of these claims.

What is claimed is:

1. A method of producing a diagnostic peptide array, wherein the method comprises:
translating one or more genomic non-protein encoding sequences into one or more peptides; and
synthesizing one or more peptides onto a substrate, wherein the one or more peptides are arranged on the substrate to be equal to or less than 3 nm from each other.

2. The method of claim 1, wherein the method is used to produce a Genome Peptide Array (GPA).

3. The method of claim 2, wherein the one or more peptides are a subset of those in the GPA.

4. The method of claim 1, wherein the diagnostic peptide array comprises 400,000 or more peptides.

5. The method of claim 1, wherein the one or more peptides is arranged on the substrate to equal about 0.5 nm to 3 nm from each other.

6. The method of claim 1, wherein the method of producing comprises up to 300 masks.

7. The method of claim 6, wherein the one or more peptides each contain 15 amino acids and all 20 amino acids are utilized in the one or more peptides.

8. The method of claim 7, wherein the one or more peptides are arranged on the substrate to be about 60 peptides per nm².

9. A diagnostic array produced by the method of claim 1.

10. A kit comprising the diagnostic peptide array of claim 9 and instructions of use.

11. A method of detecting an antibody associated with a disease of condition, comprising:
contacting a biological sample obtained from a subject with a diagnostic peptide array of claim 9; and
detecting the binding of one or more antibodies to a peptide that is associated with a disease or condition of interest, thereby detecting the antibody associated with a disease or condition.

12. The method of claim 11, wherein the biological sample is serum sample.

13. The method of claim 11, wherein the biological sample is a whole blood sample.

14. The method of claim 11, wherein the method is used to monitor efficacy of a treatment or reoccurrence.

15. The method of claim 11, wherein the method is used to diagnose a subject with cancer.

16. The method of claim 15, wherein the cancer is selected from the group consisting Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

17. The method of claim 16, wherein the breast cancer is stage 1 breast cancer.

18. The method of claim 11, wherein the method is used to diagnose Valley Fever.

19. The method of claim 11, wherein the method is used to diagnose Lyme disease.
